(19) United States
(12) Patent Application Publication
(10) Pub. No.: US 2017/0157340 A1
(43) Pub. Date: Jun. 8, 2017

(54) Drug Delivery Device with Indicator Guidance

(71) Applicant: Novo Nordisk A/S, Bagsvaerd (DK)

(72) Inventors: Mads Moeller, Hundested (DK); Mikkel Schouenborg Grubbe, Hilleroed (DK); Michael Svendsmark Hansen, Frederiksberg C (DK)

(21) Appl. No.: 15/303,856

(22) PCT Filed: Apr. 29, 2015
(86) PCT No.: PCT/EP2015/059382

§ 371 Date: Oct. 13, 2016

(30) Foreign Application Priority Data
Apr. 30, 2014 (EP) 14166513.3

(51) Int. Cl. A61M 5/50; A61M 5/36; A61M 5/315; A61M 5/24; A61M 5/145

(52) U.S. Cl. CPC A61M 5/5086; A61M 5/24; A61M 5/1452; A61M 5/31541; A61M 5/31546; A61M 5/31593; A61M 5/365; A61M 2005/2411; A61M 2005/2481; A61M 2005/2437; A61M 2005/3125

(57) ABSTRACT

A drug delivery device comprising drug expelling means for expelling an amount of drug, a number of user-oriented structures, and individual visual communication means arranged in the vicinity of at least two user-oriented structures. The device further comprises control means adapted to detect an error condition associated with each of the at least two structures, and actuate the visual communication means associated with a structure for which an error condition has been detected.

The present invention generally relates to drug delivery devices adapted to be used and operated by a patient on his or her own hand. In specific embodiments the invention relates to medical delivery devices comprising indicator means serving to guide the user during situations in which the user's attention is required.

### BACKGROUND OF THE INVENTION

In the disclosure of the present invention reference is mostly made to the treatment of diabetes by delivery of insulin or other diabetes drugs, however, this is only an exemplary use of the present invention.

Drug injection devices have greatly improved the lives of patients who must self-administer drugs and biological agents. Drug Injection devices may take many forms, including simple disposable devices that are little more than an ampoule with an injection means or they may be highly sophisticated electronically controlled instruments with numerous functions. Regardless of their form, they have proven to be great aids in assisting patients to self-administer injectable drugs and biological agents. They also greatly assist care givers in administering injectable medicines to those incapable of performing self-injections.

In particular pen-style injection devices have proven to provide an accurate, convenient, and often discrete, way to administer drugs and biological agents, such as insulin. Modern devices have become more sophisticated and often include diverse and robust functions, such as memories for remembering time and amount of last dose, as well as, in the case of insulin devices, blood glucose monitors. While pen-style injection devices are typically cylindrically shaped with a needle protruding from the most distal portion of one end of the device, some devices have other shapes with the needle no longer protruding from the most distal part of an end of the device, e.g. Innovo® and InnoLet® from Novo Nordisk A/S Bagsværd Denmark.

Typically, injection devices use a pre-filled cartridge containing the medication of interest, e.g. 1.5 or 3.0 ml of a diabetic drug (e.g. insulin or GLP-1) or growth hormone. The cartridge is typically in the form of a generally cylindrical transparent ampoule with a needle pierceable septum at one end and an opposed piston designed to be moved by the dosing mechanism of the injection device. The injection devices generally are of two types: “Durable” devices and “disposable” devices. A durable device is designed to allow a user to replace one cartridge with another cartridge, typically a new cartridge in place of an empty cartridge. In contrast, a disposable device is provided with an integrated cartridge which cannot be replaced by the user; when the cartridge is empty the entire device is discarded. Most injection devices are provided with a releasable pen cap covering the cartridge and the needle mount portion, this allowing the user to inspect the content of the cartridge by removing the cap.

Cartridge-based drug delivery devices are typically designed for use with replaceable subcutaneous needle assemblies allowing a user to mount a fresh and sterile needle before each subcutaneous injection, however, many users decide to use a needle assembly more than one time. To allow a needle assembly to be mounted in fluid communication with the cartridge through the needle-pierceable septum, the injection device is provided with a needle mount which may be formed either as part of the device or as part of the cartridge, see e.g. U.S. Pat. No. 5,693,027 and U.S. Pat. No. 6,126,646 which are hereby incorporated by reference.

In order to improve convenience, user-friendliness and provide additional features, drug delivery devices have been provided with a power source providing e.g. electrically driven expelling means and/or electronically controlled display means. For example, a drug delivery device may be provided with light sources to guide or inform a user about how to correctly use the device, see e.g. EP 2 206 529, or it may be provided with visual indicators for informing the user about a given state of the device, e.g. an error condition as disclosed in EP 2 537 546.

As indicated above, modern drug delivery devices have shown to be of great help to many people, however, for some people it still represents a challenge to correctly use and operate such a device to administer, for example, a user-set dose of a drug which has to be injected subcutaneously, e.g. insulin or other peptide drugs. This may especially be a problem in case of “non-expected” conditions, e.g. malfunctions or other error conditions which necessitate that the user takes action to resolve the issue.

Having regard to the above, it is an object of the present invention to provide drug delivery systems and devices which are configured to provide guidance helping the user to identify and resolve “non-expected” issues which may appear during operation of the device. The guidance features should be intuitive and easy to understand and should be provided in a cost-effective manner.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in accordance with a general aspect of the invention a drug delivery device is provided comprising drug expelling means for expelling an amount of drug, a number of user-oriented structures, and individual visual communication means arranged in the vicinity of at least two user-oriented structures. The device further comprises control means adapted to detect an error condition associated with each of the at least two structures, and actuate the visual communication means associated with a structure for which an error condition has been detected.

The term “error condition” also covers “normal” attention conditions such as an empty cartridge.

The term “user-oriented structure” refers to any structure which during the normal use of the device will or may require the attention of the user. A user-oriented structure may for example be in the form of a button, knob or dial which will be operated during normal setting and expelling of a dose of drug, or in the form of a coupling structure which will have to be operated when a consumable such as a subcutaneous needle or a drug cartridge have to be mounted or replaced.

The term “visual communication means” refers to any structure which can be identified by a user's eye and which can be shifted between two states, i.e. a non-active state indicating no attention required and an active state indicating attention required. The visual communication means may be light emitting e.g. based on an LED or OLED source which may be observed directly or the light may be transmitted by e.g. a light conductor. Alternatively the visual communication means may be light reflecting, e.g. electronically controlled as a LCD or a mechanical member having a colour marking being moved in and out of a viewable window.

Further, additional types of indicators may be provided, e.g. an audible “bibber” or a tactile “buzzer” which may be operated in different combinations to create specific patterns for specific conditions.

Normally, directing a user's attention to the right part of the device during an error scenario can be complicated, e.g. designing icons that are free of ambiguities are difficult and so is coming up with and presenting short text messages that are translatable and helpful. In contrast, by actuating a visual communication means (i.e. light) associated with a structure for which an error condition has been detected, the user's attention can be drawn directly towards the relevant part of the device.

In an exemplary embodiment the drug delivery device comprises a cartridge holder adapted to receive a drug-filled cartridge, the cartridge comprising an outlet and an cylindrical main body portion with an axially displaceable piston, the outlet being adapted to be arranged in fluid communication with a needle assembly comprising a hollow needle, and drug expelling means comprising a drive member adapted to engage and axially move the piston to thereby expel a dose amount of drug from a received cartridge through the outlet.

The visual communication means may comprise at least first and second visual communication means each associated with one of the following user-oriented structures: a mount for mounting a needle assembly in fluid communication with a mounted cartridge, the cartridge holder, dose setting input means for setting a dose to be expelled, and a power inlet for charging a rechargeable energy source.

The first visual communication means may be associated with the mount for mounting a needle assembly, the control means being adapted to detect one or more of the following error conditions associated with the mount and/or needle assembly: a blocked needle, no needle assembly mounted, a needle assembly incorrectly mounted, and a mounted needle assembly should be replaced.

The second visual communication means may be associated with the cartridge holder, the control means being adapted to detect one or more of the following error condition associated with the cartridge holder: too much air in the cartridge, cartridge too old (i.e. as calculated from when the cartridge was inserted in the cartridge holder), cartridge empty, no cartridge mounted, cartridge mounted incorrectly, and user attempts to set a dose larger than the dose amount remaining in the cartridge.

At least one visual communication means comprises a light emitter which may be arranged in combination with a light guide for transmitting light from the light emitter to a corresponding light emitting area.

The drug delivery device may further comprise an electronically controlled display, the display and control means being adapted to provide additional information related to the detected error condition having caused actuation of a given visual indicator.

As used herein, the term “drug” is meant to encompass any drug-containing flowable medicine capable of being passed through a delivery means such as a cannula or hollow needle in a controlled manner, such as a liquid, solution, gel or fine suspension. Representative drugs include pharmaceuticals such as peptides (e.g. insulins, insulin containing drugs, GLP-1 containing drugs as well as derivates thereof), proteins, and hormones, biologically derived or active agents, hormonal and gene based agents, nutritional formulas and other substances in both solid (dispensed) or liquid form. In the description of the exemplary embodiments reference will be made to the use of insulin containing drugs. Correspondingly, the term “subcutaneous” infusion is meant to encompass any method of transcutaneous delivery to a subject.

### BRIEF DESCRIPTION OF THE DRAWINGS

In the following embodiments of the invention will be further described with reference to the drawings, wherein

FIG. 1 shows schematically an embodiment of a drug delivery device,

FIGS. 2A-2D show in different states a drug delivery device comprising light emitting areas,

FIG. 3 shows a first embodiment of a drug delivery device platform, and

FIGS. 4 and 5 show a second embodiment of a drug delivery device platform.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The term “assembly” does not imply that the described components necessarily can be assembled to provide a unitary or functional assembly during a given assembly procedure but is merely used to describe components grouped together as being functionally more closely related.

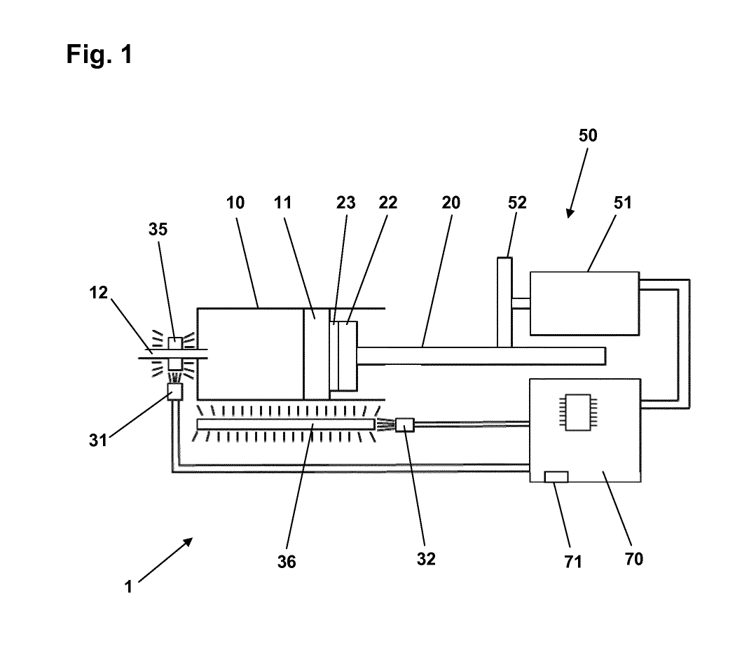

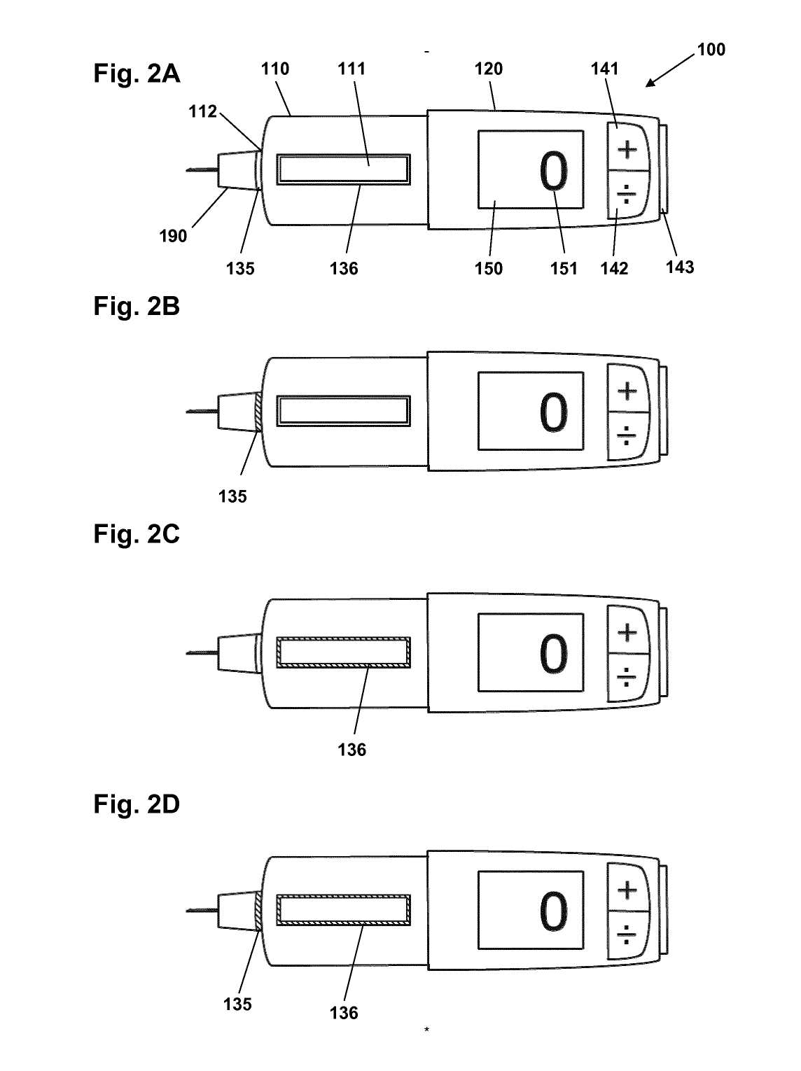

With reference to FIG. 1 a schematic embodiment of a motorized drug delivery device 1 comprising a dynamic guidance and attention system using light indicators will be described, the device in addition to a display (see FIG. 2A) being provided with electronically controlled visual markers arranged in the vicinity of different attention areas of the device.

More specifically, the motorized drug delivery device 1 comprises a main portion in which an expelling assembly 50 is arranged, and a cartridge holder portion adapted to receive and hold an exchangeable drug-filled cartridge 10, the cartridge comprising an axially displaceable piston 11 and a distal outlet 12 associated with coupling means allowing a needle assembly to be mounted. In the shown embodiment the expelling assembly comprises a piston rod 20 adapted to engage and move forward a cartridge piston to thereby expel an amount of drug, the piston rod being driven by an electronically controlled motor 51 via a gear assembly 52. The piston rod comprises a distal piston rod washer 22 in which a sensor 23, e.g. a force sensor, is arranged. The device further comprises first and a second light emitters 31, 32 directing light into first and a second light guides 35, 36 each having a light exit area providing a visual light indicator when light from a light emitter is transmitted there through. In the shown embodiment the first light indicator is arranged in the vicinity of the needle mount and the second light indicator is arranged along a portion of the cartridge holder. The device further comprises electronic controller circuitry 70 adapted to control operation of the motor in order to move the piston rod in a distal or proximal direction. A rechargeable power source (“battery”) with a power inlet 71 is associated with the controller circuitry. The controller circuitry is further adapted to receive input from user input means (see below) as well as from one or more sensors, e.g. the shown piston rod sensor, just as the controller circuitry is adapted to control a display and the light emitters in accordance with detected operational conditions. The piston rod sensor may be used to detect piston rod engagement with the cartridge piston when the piston rod is forwarded after cartridge exchange, however, the signals from the sensor may also be analysed by the processor circuitry to determine specific conditions, e.g. a blocked needle or the amount of air in the cartridge, see patent application EP 14183758.3 which is hereby incorporated by reference.

The device further comprises a rechargeable energy source. In the shown embodiment the light emitters are arranged apart from the controller circuitry, however, the light emitters may be arranged centrally on a common PCB, the light being guided to the indicator areas by correspondingly longer light guides.

Turning to FIGS. 2A-2D an exemplary drug delivery device comprising two light indicators is shown schematically. As described above with reference to FIG. 1, FIG. 2A shows a motorized drug delivery device 100 comprising a main portion 120 in which an expelling assembly is arranged, and a cartridge holder portion 110 adapted to receive and hold an exchangeable drug-filled cartridge. The cartridge holder portion comprises a distal opening 112 and a window 111 allowing a user to visually inspect the content of a loaded cartridge just as the actual position of the cartridge piston can be observed. A needle assembly 190 is mounted in fluid communication with a loaded cartridge. The main portion comprises user input means in the form of a pair of dose setting buttons 141, 142 allowing a user to set and adjust a dose of drug to be expelled, as well as a dose release button 143 arranged at the proximal end of the device. A display 150 shows the currently set dose 151. The display may be controlled to provide further information to a user, e.g. the dose numeral may count down during dose expelling, just as the display may comprises indicators for e.g. battery condition, error conditions, and time. For example, symbols in the display may be used to further specify what kind of error condition is associated with a given actuated visual indicator.

In the shown embodiment a first ring-formed visual indicator 135 is arranged corresponding to where a needle assembly is to be mounted, and a second frame-formed visual indicator 136 is arranged corresponding to the cartridge holder inspection window 111. In case two opposed inspection windows are provided each may be provided with a visual indicator. It should be noted that the needle assembly mount per se may be formed as part of the replaceable cartridge and thus not the device. Correspondingly, the user-oriented structure associated with such a needle mount would be the opening 112 through which a cartridge-mounted needle mount would project. According to aspects of the invention, the first visual indicator is operated to direct a user to problems or error conditions associated with the needle and the second visual indicator is operated to direct a user to problems or error conditions associated with the cartridge. The light emitters may be operated to provide constantly lighted indicators or to provide one or more flashing patterns which may be used for specific conditions. The first and second light indicators or additional light indicators may be arranged in the vicinity of other structures. For example, a light indicator may be associated with the dose setting buttons and indicate when the user attempts to set a too large dose, e.g. larger than the remaining amount of drug or larger than a pre-set maximum dose. A light indicator may be arranged in the vicinity of a power inlet indicating when the device needs to be recharged. Further, additional types of indicators may be provided, e.g. an audible “bibber” or a tactile “buzzer” which may be operated in different combinations to create specific patterns for specific conditions.

By providing visual indicators physically arranged corresponding to a specific location of concern, the problems associated with small or difficult-to-understand error messages shown in a convention display, e.g. the shown dose setting display, can be removed or reduced.

For example, conditions detected for the needle could be one or more of the following: Blocked needle, no needle mounted, needle incorrectly mounted, or needle should be replaced. The conditions detected for the cartridge could be one or more of the following: Too much air in the cartridge, cartridge too old (i.e. as calculated from when the cartridge was inserted in the cartridge holder), cartridge empty, no cartridge mounted, cartridge incorrectly mounted, or the user attempts to set a dose larger than the dose amount remaining in the cartridge. Whether a given amount of detected in the cartridge is considered “too much air” may be related to the size of a currently set dose, e.g. the larger the dose the more air may be acceptable.

Some of the above conditions may be detected by “simple” sensors, e.g. detecting whether or not a needle or cartridge is (correctly) mounted, whereas others may be detected by analysing output from more advanced sensors, e.g. determining a blocked needle condition or the amount of air in the cartridge based on output from the piston rod sensor, see above. Indeed, a given analysis for detecting e.g. a blocked needle condition may detect the same condition when no needle is mounted, however, which of the two conditions is at issue should be apparent to the user. Further, “blocked needle” may include conditions in which the needle is blocked to a certain degree, i.e. not fully.

Turning to FIGS. 2A-2D, FIG. 2A shows the drug delivery device with the light indicators off, FIG. 2B shows the drug delivery device with the needle light indicator 135 on, FIG. 2C shows the drug delivery device with the cartridge light indicator 136 on, and FIG. 2D shows the drug delivery device with both the needle and the cartridge light indicators 135, 136 on.

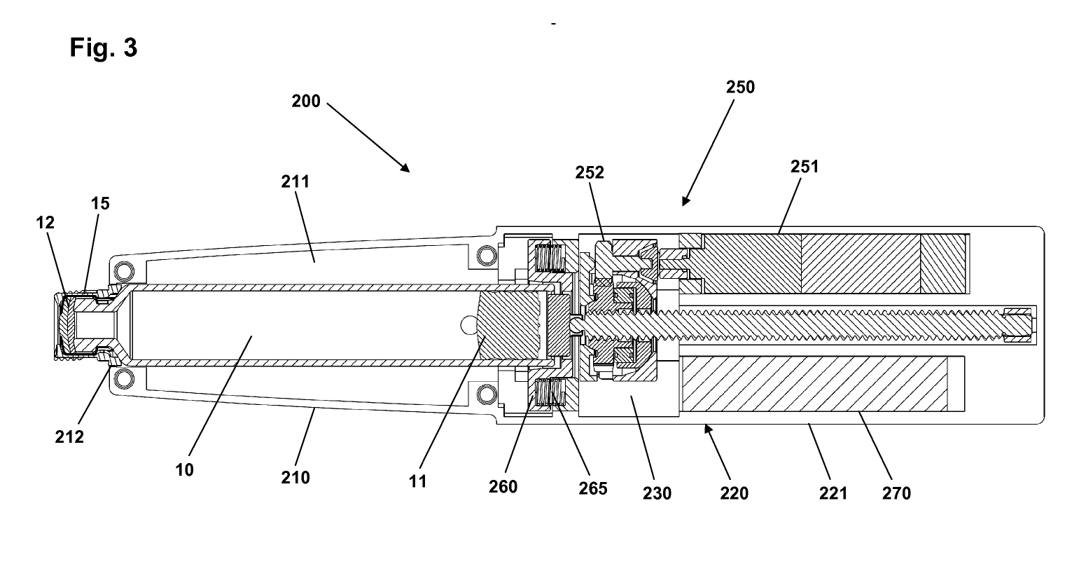

Turning to FIG. 3 a first embodiment 200 of a drug delivery device suitable as a platform for embodiments of the present invention will be described. More specifically, the device comprises a cap part (not shown) and a main part having a proximal body or drive assembly portion 220 with a housing 221 in which a motorized drug expelling assembly 250, electronic controller circuitry 270 and an electric power source are arranged, and a distal cartridge holder portion 210 with a compartment 211 in which a drug-filled cartridge 10 is arranged and retained in place. The cartridge comprises a generally cylindrical main portion with an axially displaceable piston 11 and a distal outlet portion 12 comprising a needle-penetrable septum. The cartridge is further provided with distal coupling means in the form of a needle hub mount 15 having, in the shown example, an external thread adapted to engage an inner thread of a corresponding hub of a needle assembly. The cartridge may for example contain an insulin, a GLP-1 or a growth hormone formulation. The device further comprises dose setting means allowing a user to set a dose of drug to be expelled as well as a display showing the set dose, e.g. as shown in FIG. 2A.

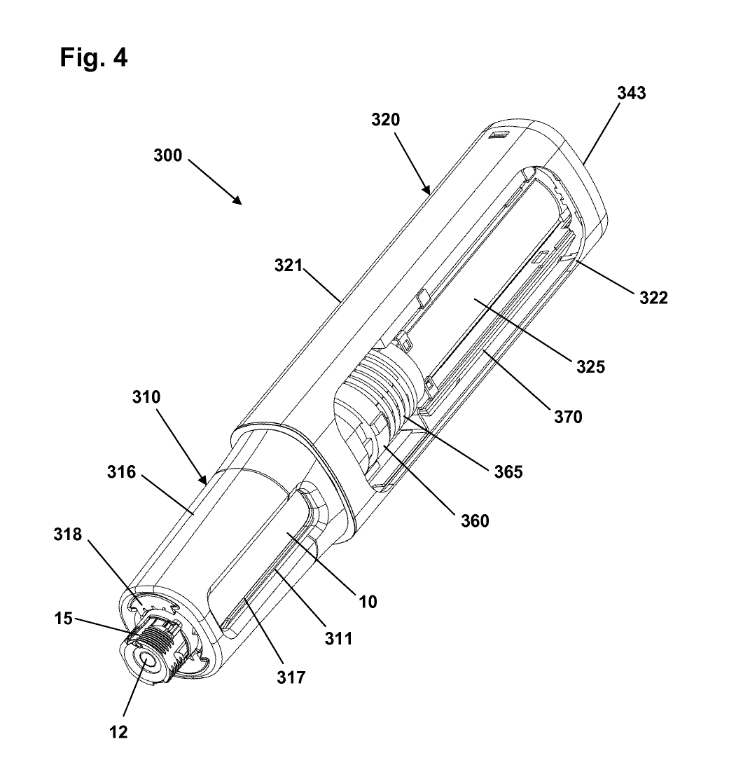

In the shown embodiment the device is designed to be loaded by the user with a new cartridge through a distal receiving opening 212 in the cartridge holder assembly, the cartridge holder comprising closure means (not shown) operatable by a user between an open position in which a cartridge can be inserted respectively removed, and a closed position in which an inserted cartridge is held in place. The closure means may be of the same type as described with respect to FIG. 4 below. In order to axially position the cartridge, the device comprises a seat member 260 adapted to receive the proximal end of the cartridge, the seat member being biased in the proximal direction by springs 265 thereby forcing the cartridge into contact with the closure means.

When incorporating an embodiment of the present invention in the platform device of FIG. 3, two light emitters, e.g. two LEDs, could be arranged in the space 230 opposite the gearbox, the LEDs being mounted directly on a portion of the PCB onto which the other components of the electronic circuitry are arranged. The light conductors and the light exit areas could be formed integrally with a component of the cartridge holder, e.g. by 2K injection moulding, the light conductors being made from LISA plastic, e.g. Makrofol® which is a coloured light-collecting and light-conducting polycarbonate-based material sold by Bayer MaterialScience AG, Leverkusen, Germany. In this way light indicator guidance means could be incorporated in an otherwise electronically controlled drug delivery device in a cost-effective way.

Turning to FIG. 4 a second embodiment 300 of a drug delivery device suitable as a platform for embodiments of the present invention will be described. More specifically, the device comprises a cap part (not shown) and a main part having a proximal body or drive assembly portion with a housing 321 in which a drug expelling mechanism and associated electronics 370 are arranged, and a distal cartridge holder assembly 310 forming a compartment in which a drug-filled transparent cartridge 10 can be arranged and retained in place, the cartridge holder assembly comprising a pair of opposed inspection openings 311. The housing comprises an opening 322 adapted to receive a display frame member (not shown) in which a LCD as well as user input keys are mounted, e.g. as shown in FIG. 2A. With the frame member removed, it can be seen that the device comprises a generally tubular chassis member 325, in which a generally cylindrical expelling assembly is mounted (see below). The device further comprises a control assembly 370, a bias assembly comprising a bias member 360 and a spring 365, and a proximal release button 343. A pair of dose setting input keys (not shown) serves to manually set a desired dose of drug shown in the LCD and which can then be expelled when the release button 90 is actuated. The device is designed to be loaded by the user with a new cartridge through a distal receiving opening in the cartridge holder assembly.

The cartridge 10 comprises a cylindrical body portion, a distal outlet portion 12 with a distal needle-penetrable septum, and an axially displaceable piston having a proximal surface allowing a piston driver forming part of the expelling mechanism (see below) to engage the piston. The cartridge may for example contain an insulin, a GLP-1 or a growth hormone formulation. The cartridge is provided with distal coupling means in the form of a needle hub mount 15 having, in the shown example, combined thread and bayonet coupling means, each being adapted to engage an inner thread or bayonet coupling means of a corresponding hub of a needle assembly. The shown exemplary hub mount further comprises a circumferential flange with a number of distally facing pointed projections serving as a coupling means for the cartridge holder assembly as will be described in more detail below. A hub mount of the shown type is described in U.S. Pat. No. 5,693,027. Alternatively the needle hub mount may be formed as part of the cartridge holder, e.g. in the form of a “split” hub mount having two parts arranged on each side of the gripping shoulders.

As shown, the cartridge holder assembly 310 has the same general appearance as a traditional cartridge holder which is detachably coupled to the housing by e.g. a threaded coupling or a bayonet coupling and into which a new cartridge can be received as well as removed through a proximal opening, i.e. it comprises no additional user operated release or locking means. Instead, what appears merely to be the cartridge holder per se is in fact user operated coupling means in the form of an outer rotatable tubular actuation sleeve 316 operated by the user to control movement of cartridge holding means in the form of an inner cartridge holder member 317 to thereby open and close gripping shoulders 318 configured to grip and hold a cartridge. More specifically, each gripping shoulder is provided with a plurality of gripping teeth spaced circumferentially to provide a plurality of gaps, each tooth having a triangular configuration with a proximally oriented pointed end, thereby creating a plurality of gaps having a distally oriented pointed configuration, this allowing the above-described distally facing pointed projections on the cartridge to be received between the teeth to thereby serve as a gripping means when the cartridge holding means has been moved into engagement with the cartridge. In this way an easy-to-use front loaded drug delivery device is provided which appears as a traditional rear loaded device and which is also actuated by rotational movement to mount and remove a cartridge, the resemblance providing for ease of acceptance and adaptation among users accustomed to traditional types of rear loaded drug delivery devices.

When it is time to mount a new cartridge the outer tube member 316 is rotated e.g. 90 degrees by which action the gripping shoulders 318 are moved distally and slightly outwards, this allowing the mounted cartridge to be removed. For ease of operation the cartridge may be moved distally a certain distance as the shoulders are moved, e.g. by engagement with arms forming the gripping shoulders and/or by additional spring means providing a biasing distally directed force (see below). Depending on the design of the locking and actuation mechanism the gripping shoulders may be able to be left in the open position or they may be retracted automatically as the outer tube member is rotated backwards by return spring means. Whether or not a spring is provided the cartridge holder may be provided with locking means allowing the outer tube member to be securely parked in either the open or closed position, e.g. by a rotational snap lock. When a new cartridge is inserted the drive expelling means has to be in a state allowing a new cartridge with a proximally positioned piston to be inserted. An exemplary embodiment providing this functionality will be described below.

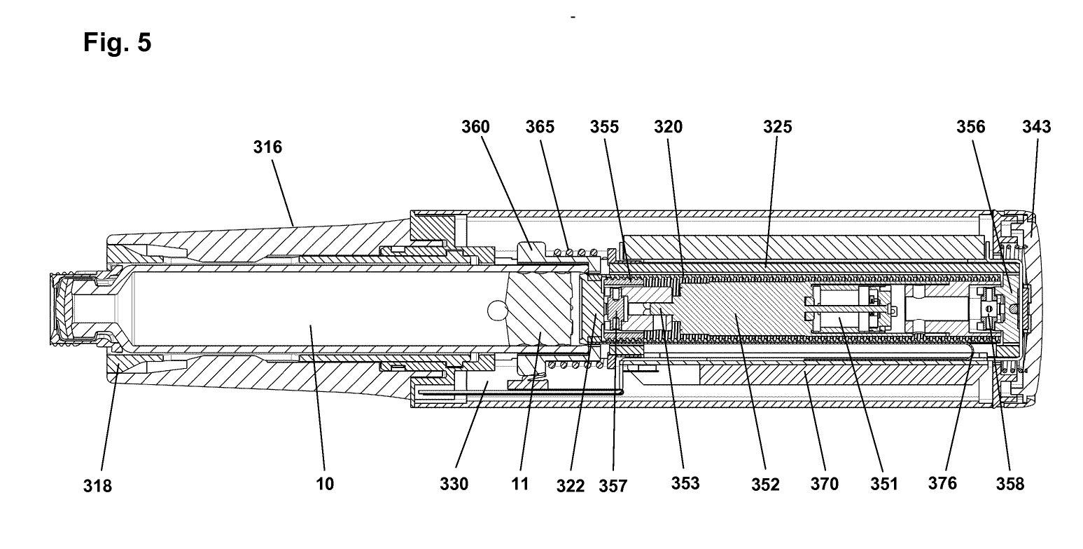

Turning to FIG. 5 a cross-sectional view of the drug delivery device 300 of FIG. 4 is shown with a mounted cartridge 10 and with the piston tube 320 (see below) in a fully retracted position. More specifically, the actuation sleeve 316 has been rotated to its operational position and the cartridge holder gripping shoulders 318 have been retracted to their closed position thereby retracting the cartridge to its fully inserted position, thereby also moving the bias member 360 proximally against the bias of the spring 365. In the shown embodiment a cartridge switch 375 is hereby being actuated, this providing a signal to the device controller that two actions can be assumed to have taken place: (i) a cartridge has been inserted and (ii) the cartridge holder has been closed, this initiating that the drive head is moved distally into contact with the cartridge piston. In the shown embodiment it is contemplated that detection of contact between the drive head and the piston is detected by electronic sensor means arranged in the drive head, e.g. using proximity detection as disclosed in WO 2013/144152.

FIG. 5 also shows the expelling assembly in greater detail. More specifically, the expelling assembly is in the form of a motor-in-piston assembly comprising an interior motor and gearbox drive assembly mounted axially and rotationally locked to the proximal end of the chassis, and an outer axially displaceable piston tube 320 with a distal drive head 322 adapted to engage the piston 11 of a loaded cartridge, the piston tube comprising a number of guide projections adapted to non-rotationally engage corresponding guide means of the chassis.

The motor-gear drive assembly comprises a tubular main portion composed of a proximal motor assembly 351 and a distal gearbox assembly 352 having a rotatable drive shaft 353 defining a z-axis of rotation. The assembly further comprises a distal cylindrical drive member 355 having an outer thread adapted to be arranged in engagement with the piston drive tube inner thread. At the proximal end a disc-formed chassis connector 356 is arranged. In the shown embodiment the drive assembly is provided with flexible joints in the form of a distal universal joint 357 arranged between the drive shaft and the drive member and a proximal universal joint 358 arranged between the motor assembly proximal portion and the chassis tube proximal portion. A corresponding drive assembly is described in greater detail in patent application EP 14166859.0, which is hereby incorporated by reference.

A number of further details can be seen in FIG. 5. The release button 343 is received in the housings proximal opening with a spring providing a proximally directed biasing force on the button. A flexible ribbon 376 with a plurality of conductors is arranged with a U-bend between the electronics portion 370 and the sensors (not shown) arranged in the piston head, this allowing the piston tube and piston head to travel axially with the U-bend moving correspondingly.

When incorporating an embodiment of the present invention in the platform device of FIGS. 4 and 5, two light emitters, e.g. two LEDs, could be arranged in the vicinity of the switch 375, the LEDs being mounted directly on a portion of the PCB onto which the other components of the electronic circuitry are arranged. The light conductors and the light exit areas could be formed integrally with a component of the cartridge holder, e.g. the actuation sleeve 316, as described above.

In the above description of the preferred embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A drug delivery device comprising:
drug expelling structure for expelling an amount of drug,
a number of user-oriented structures,
individual visual communication structure arranged in the vicinity of at least two user-oriented structures, and
control structure adapted to:
detect an error condition associated with each of the at least two structures, and
actuate the visual communication structure associated with a structure for which an error condition has been detected.

2. A drug delivery device as in claim 1, comprising:
a cartridge holder adapted to receive a drug-filled cartridge, the cartridge comprising an outlet and an cylindrical main body portion with an axially displaceable piston, the outlet being adapted to be arranged in fluid communication with a needle assembly comprising a hollow needle, and
drug expelling structure comprising a drive member adapted to engage and axially move the piston to thereby expel a dose amount of drug from a received cartridge through the outlet.

3. A drug delivery device as in claim 2, wherein the visual communication structure comprises at least first and second visual communication structure each associated with one of the following user-oriented structures:
a mount for mounting a needle assembly in fluid communication with a mounted cartridge,
the cartridge holder,
dose setting input structure for setting a dose to be expelled, and
a power inlet for charging a rechargeable energy source.

4. A drug delivery device as in claim 3, wherein the first visual communication structure is associated with the mount for mounting a needle assembly, the control structure being adapted to detect one or more of the following error conditions associated with the mount and/or needle assembly:
blocked needle,
no needle assembly mounted,
needle assembly incorrectly mounted, and
mounted needle assembly should be replaced.

5. A drug delivery device as in claim 3, wherein the second visual communication structure is associated with the cartridge holder, the control structure being adapted to detect one or more of the following error condition associated with the cartridge holder:
too much air in the cartridge,
cartridge too old,
cartridge empty,
no cartridge mounted,
cartridge mounted incorrectly, and
user attempts to set a dose larger than the dose amount remaining in the cartridge.

6. A drug delivery device as in claim 1, wherein at least one visual communication structure comprises a light emitter.

7. A drug delivery device as in claim 1, wherein at least one visual communication structure comprises a light emitter in combination with a light guide for transmitting light from the light emitter to a corresponding light emitting area.

8. A drug delivery device as in claim 1, further comprising an electronically controlled display, the display and control structure being adapted to provide additional information related to the detected error condition having caused actuation of a given visual indicator.
